(19) United States
(12) Patent Application Publication
(10) Pub. No.: US 2015/0150831 A1
(43) Pub. Date: Jun. 4, 2015

(54) SKIN CLEANSING COMPOSITION WITH ENHANCED RHEOLOGICAL PROPERTIES

(71) Applicant: The Dial Corporation, Scottsdale, AZ (US)

(72) Inventors: Kanani Guerra, Phoenix, AZ (US); Nancy D. Rodgers, Chandler, AZ (US); Jared Schoepf, Phoenix, AZ (US)

(73) Assignee: The Dial Corporation, Scottsdale, AZ (US)

(21) Appl. No.: 14/096,296

(22) Filed: Dec. 4, 2013

(51) Int. Cl. A61K 31/14

(52) U.S. Cl. CPC A61K 31/14

(57) ABSTRACT

Materials and apparatus are provided for a skin cleansing composition with enhanced rheological properties. The skin cleansing composition includes a number of surfactants. The skin cleansing composition further includes at least one cationic antimicrobial active. The skin cleansing composition also includes a quaternary amine salt. Ratios of the components of the skin cleansing composition which provide the enhanced rheological properties are also provided. The skin cleansing composition also includes an aqueous medium in which the number of surfactants, the at least one cationic antimicrobial active and the quaternary amine salt are distributed.

### FIELD OF THE INVENTION

The present invention relates to a composition with enhanced rheological properties, and more specifically relates to a skin cleansing composition with a cationic antimicrobial active ingredient which exhibits enhanced rheological properties for increased consumer appeal.

### BACKGROUND OF THE INVENTION

The feel of a skin cleansing composition may increase consumer appeal. Several commercially available formulations with good skin feel and lather properties may employ an anionic antimicrobial active ingredient. Some antimicrobial actives may be cationic antimicrobial actives, and are in formulations which may include a number of cationic surfactants, moisturizers, and other components of skin cleansing compositions.

Accordingly, it is desirable to have a skin cleansing composition which employs a cationic antimicrobial active ingredient. In addition, it is desirable to have a skin cleansing composition which has rheological properties which make it appealing to a consumer. The present disclosure describes a skin cleansing composition which uses a cationic antimicrobial active ingredient with enhanced rheological properties. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A skin cleansing composition with enhanced rheological properties contains a number of surfactants, at least one cationic antimicrobial active and a quaternary amine salt. A ratio of quaternary amine salt to a combined mass of non-aqueous components in the skin cleansing composition is within the range of 1:4 to 1:15, a ratio of quaternary amine salt to a thickening agent in the skin cleansing composition is within the range of 1:2 to 3:2, and the quaternary amine salt comprises between 0.5 to 2.0 weight percent of the skin cleansing composition. The skin cleansing composition also includes an aqueous medium in which the number of surfactants, the at least one cationic antimicrobial active and the quaternary amine salt are distributed.

A skin cleansing product with enhanced rheological properties contains a skin cleansing composition, wherein the skin cleansing composition contains a cationic antimicrobial active, a number of surfactants, a quaternary amine salt wherein a ratio of quaternary amine salt to a combined mass of non-aqueous components is within the range of 1:4 to 1:15; the skin cleansing composition also contains an aqueous medium in which the number of surfactants, the at least one cationic antimicrobial active and the quaternary amine salt are distributed. The skin cleansing product is housed within a container capable of dispensing the skin cleansing composition.

A skin cleansing composition with enhanced rheological properties contains a cationic antimicrobial active, a number of surfactants, and an aqueous medium in which the cationic antimicrobial active and the number of surfactants are distributed. The skin cleansing product possesses a controlled shear rate slope between −840 and −532 centipoise per second at 25 degrees Celsius (C), and also loses at least 40% of its original viscosity during the second interval of a three interval thixotropic test, under a shear stress of 100 s⁻¹, at 25 degrees C.

### BRIEF DESCRIPTION OF THE DRAWINGS

The present invention will hereinafter be described in conjunction with the following drawing figures, wherein like numerals denote like elements, and

FIG. 1 is a diagram of an exemplary container dispensing a skin cleansing composition according to the principles described herein.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The lather and skin feel properties may be valuable to consumer acceptance of a skin cleansing composition. Such lather and skin feel properties may be a factor in the market acceptance and value of a skin cleansing composition, which may then act independently of the ability of such a skin cleansing composition to effectively cleanse the skin, or the ability of an antimicrobial active ingredient to effectively combat microbes present on the skin.

Rheology may be the study of the flow of matter. Thus, the rheological properties may determine the lather and skin feel properties of a skin cleansing composition. The rheological properties of a skin cleansing composition may be valuable to its consumer acceptance. Such properties may include a viscosity of a skin cleansing composition, and how a viscosity of a skin cleansing composition changes in response to environmental stimuli. For example, a viscosity may decrease in response to added force, and may exhibit some variation in the rate at which it returns to the original viscosity upon the removal of such a force.

Attaining the rheological properties that may be desirable to attain consumer acceptance of a skin cleansing formulation while using a cationic antimicrobial active remains an outstanding challenge in the field, where a general push towards cationic antimicrobial actives may have resulted in a change in the composition of many of the elements of skin cleansing compositions, including surfactants, preservatives, thickening agents, or combinations thereof.

Accordingly, the principles described herein provide rheological properties desirable to attain consumer acceptance of a skin cleansing formulation, as well as providing a skin cleansing composition utilizing a cationic antimicrobial active ingredient that exhibits these rheological properties.

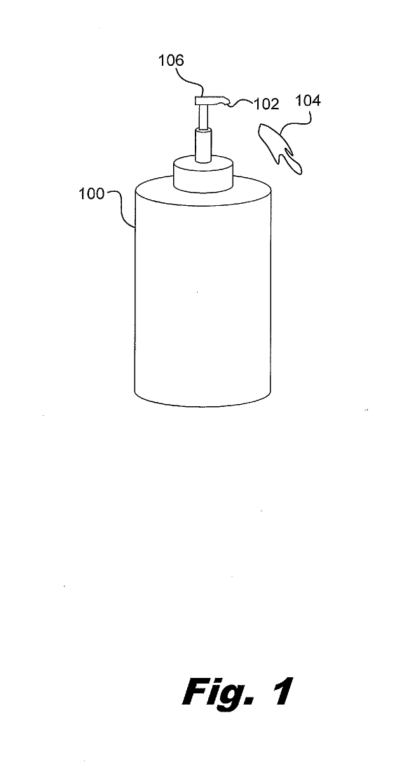

Turning now to the figures, FIG. 1 is a diagram of an exemplary container (100) dispensing a skin cleansing composition (104) according to the principles described herein. The container (100) may be equipped with an opening (102) capable of dispensing the skin cleansing composition (104). Such an opening (102) may provide the ability to dispense the skin cleansing composition (104) to an area of the skin. In this example, the skin cleansing composition may be held within a container (100) equipped with a plunger type dispensing mechanism (106) to dispense the skin cleansing composition through the opening (102). The skin cleansing composition (104) held within the container may be applied to the skin of the user in order to cleanse the region of the skin to which the skin cleansing composition (104) is applied. The container shown in FIG. 1 is exemplary, and does not represent all types of dispensers which may be used to dispense the skin cleansing composition (104) from the container (100) in which it is held. Another type of dispenser (100) may include a simple opening allowing for the dispensing of the skin cleansing composition (104), which dispenser (100) may have a cap.

The skin cleansing composition (104) may include a cationic antimicrobial active. Such an ingredient may be positively charged and may be capable of killing bacteria. Examples of such cationic antimicrobial actives may include benzethonium chloride, benzalkonium chloride, or cationic antimicrobial peptides.

The skin cleansing composition (104) may include a number of surfactants. A surfactant may have a hydrophobic end and a hydrophilic end. The hydrophobic end may allow the surfactant to interact with uncharged molecules, such as oils. The hydrophobic end may be a hydrocarbon, which may be linear, branched, cyclic or aromatic. The hydrophilic end may facilitate the interaction of the molecule with charged or polar molecules, such as water. The hydrophilic end may be used to classify surfactants, which may be anionic, cationic, nonionic, or zwitterionic. Anionic surfactants may have a negatively charged hydrophilic end, which may be present as a sulfate, sulfonate, carboxylate or the like; anionic surfactants may be sensitive to water hardness. Cationic surfactants may be those that have a positively charged hydrophilic end, such as a quaternary amine. Nonionic surfactants may have a hydrophilic end which may be charge neutral, such as an ethoxylate or poly-ol; such surfactants may not be sensitive to water hardness. Zwitterionic or amphoteric surfactants may have both a positive and negative charge on their hydrophilic ends, such as amine oxides. In some examples, the number of surfactants may include lauramine oxide, lauramidopropylamine oxide, lauramidopropyl betaine, lauryl betaine, cocamidopropyl betaine, or combinations thereof. While specific reference has been made to certain surfactants, the number of surfactants in the skin cleansing composition (104) may include any type, combination or mixture of surfactants. For example, the number of surfactants may include a single nonionic surfactant. Another example may have the number of surfactants be a blend of cationic and zwitterionic surfactants.

The skin cleansing composition (104) may also include a quaternary amine salt. Such a quaternary amine salt may act as a conditioning agent. Such a quaternary amine salt may act as a thickening agent. A quaternary amine salt may also otherwise modify the rheological properties of the skin cleansing composition (104). A quaternary amine salt may have at least one nitrogen atom, which has a positive charge as it is covalently bound to four other atoms, which may be any atom, such as carbon, oxygen or silicon. A quaternary amine salt may also have a counterion, which may have a charge that is opposite the charge of the amine-bearing functionality.

Quaternary amine salts, which may have the chemical structure

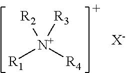

may also be present in the skin cleansing composition (104). Examples of quaternary amine salts may be when R₁, R₂ and R₃ are methyl (—CH₃), and R₄ is lauryl (—(CH₂)₁₁CH₃), myristyl (—(CH₂)₁₃CH₃), cetyl (—(CH₂)₁₅CH₃) or stearyl (—(CH₂)₁₇CH₃), and X⁻is Cl⁻, which may be called lauryl(trimethyl)ammonium chloride, myristyl(trimethyl)ammonium chloride, cetrimonium chloride and stearyl(trimethyl)ammonium chloride, respectively. Other examples of quaternary amine salts may involve linear, branched, cyclic, aromatic, non-hydrocarbon, or combinations thereof as the R₁, R₂, R₃ and R₄ substituents. The indicated charges are examples, and are not intended to limit the scope, applicability or configuration of the skin cleansing composition (104) in any way. Additional examples of quaternary amine salts may have a net negative charge on the amine-bearing component, accompanied by a cationic counterion.

The skin cleansing composition (104) may have certain rheological properties, which may be measured by a rheometer. A rheometer may be a device capable of measuring a number of rheological properties, which may assay rheological properties of a sample in a number of ways. For example, some rheometers may assay a sample using a capillary tube; another type of rheometer may assay a sample positioned between two plates, and still another type of rheometer may assay a sample using various turbine attachments in a container of the material to be tested. The values presented herein were produced on an Anton Paar™ rheometer.

Rheological properties may be the properties of a liquid, gel, or other flowable material, and may be used to characterize the flow properties of the material. For example, viscosity may be a rheological property. Another example of a rheological property may be the changes to viscosity and rates of said changes to viscosity in response to applied force, based on the nature of that force.

A rheological property that may be used to indicate consumer acceptance of a skin cleansing composition (104) is the performance in a controlled shear rate (CSR) test. The CSR test may measure the extent to which a material being tested changes its viscosity in response to the application of a controlled force. The results of the CSR test may be expressed as the slope of the best fit line, which may be fit to the data points, wherein a negative slope indicates that the viscosity of the material being tested decreased in response to applied force (sheer thinning), a positive slope indicates that the viscosity of the material being tested increased in response to an applied force (sheer thickening or dilatant), and a slope of zero indicates that the material being tested did not change viscosity in response to an applied force.

The skin cleansing composition (104) discussed herein may have CSR slopes in the range of −840 to −532 centipoise per second. For example, the skin cleansing composition (104) may have CSR slopes in the range of −840 to −685 centipoise per second. These values may represent ranges which may make a skin cleansing composition (104) appealing to a consumer.

Another rheological property that may indicate consumer acceptance of a skin cleansing composition (104) is the performance in a three interval thixotropic test (3ITT). Such a test may be conducted as follows: (1) first, the material to be tested is subjected to low shear stress for sixty seconds, wherein the shear rate is approximately 0.1 s⁻¹; (2) second, the material to be tested is subjected to high shear stress for ten seconds, wherein the shear rate is approximately 100 s⁻¹; (3) third, the material to be tested is returned to low shear conditions for 20 seconds, with a shear rate of approximately 0.1 s⁻¹. The viscosity of the material being tested may be monitored throughout the test.

A desirable property of a skin cleansing composition (104) may be for the viscosity of a material to decrease by at least 40 percent during the second interval (2) of the three interval thixotropic test (3ITT). In some examples, the skin cleansing composition (104) may have a viscosity decrease of at least 47 percent during the second interval (2) of the three interval thixotropic test (3ITT).

The skin cleansing composition (104) may also comprise a thickening agent. A thickening agent may be an ingredient which is added to the skin cleansing composition (104) to increase its viscosity. An example of a thickening agent may be PEG-120 Methyl Glucose Dioleate, or other polyethylene-glycol based agents. Another example of a thickening agent may be hydromethylcellulose.

In some examples, the skin cleansing composition (104) may include ingredients in amounts relative to one another, represented as ratios. These ratios of ingredients may exhibit preferable rheological properties, as noted above. In other words, the ratios of ingredients of the skin cleansing composition (104) may allow the use of a cationic antimicrobial active ingredient, and may also maintain the rheological properties which improve consumer appeal. One such ratio may be the ratio of quaternary amine salt to thickening agent. Another such ratio may be the ratio of quaternary amine salt to a combined mass of the non-aqueous components in the skin cleansing composition (104). A third such ratio may be the weight percentage of a quaternary amine salt in the skin cleansing composition (104). A fourth such ratio may be the combined weight of the non-aqueous components of the skin cleansing composition (104) as a percentage of the weight of the skin cleansing composition (104).

The ratio of a quaternary amine salt to thickening agent in the skin cleansing composition (104) may be within the range of 1:2 to 3:2. For example, the ratio of a quaternary amine salt to thickening agent is with the range of 1:2 to 23:20, and more specifically within the range of 3:4 to 23:30.

The ratio of a quaternary amine salt to a combined mass of the non-aqueous components in the skin cleansing composition (104) may be within the range of 1:4 to 1:15. For example, the ratio of a quaternary amine salt to a combined mass of the non-aqueous components in the skin cleansing composition (104) may be within the range 1:5 to 1:12, and more specifically within the range of 2:11 to 1:11.

The weight percentage of a quaternary amine salt in the skin cleansing composition (104) may also have a bearing on the rheological properties of the skin cleansing composition (104). The weight percentage of a quaternary amine salt in the skin cleansing composition (104) may be within the range of 0.5 to 2.29. For example, the weight percentage of a quaternary amine salt in the skin cleansing composition (104) is within the range of 0.75 to 2.0.

The combined weight of the non-aqueous components as a percentage of the weight of the skin cleansing composition (104) may also indicate the rheological properties of a skin cleansing composition (104). For the skin cleansing composition (104), the combined weight of the non-aqueous components may be within the range of 6 to 10 percent of the weight of the skin cleansing composition (104). For example, the combined weight of the non-aqueous components of the skin cleansing composition (104) may be within the range of 7.5 to 9.0 percent of the weight of the skin cleansing composition (104).

In some examples, the skin cleansing composition (104) may include other ingredients to increase consumer appeal, such as a moisturizer, a foaming agent, preservative, dye, fragrance or combinations thereof. Moreover, in some examples, the skin cleansing composition (104) may be a rinse-off formulation.

The skin cleansing composition (104) may also comprise an aqueous medium in which the number of surfactants, the at least one cationic antimicrobial active and the quaternary amine salt are distributed. Any other ingredients in the skin cleansing composition (104) may be similarly distributed in said aqueous medium.

The skin cleansing compositions (104) using a cationic antimicrobial active which may be disclosed herein are unique with respect to their rheological performance, which may provide for increased consumer acceptance. Table (1) below presents ranges of various components of an exemplary skin cleansing composition (104) as described herein. In Table (1), benzethonium chloride may be the cationic antimicrobial active, and cetrimonium chloride may be the quaternary amine salt. PEG-120 Methyl Glucose Dioleate may be used as the thickening agent, and the surfactants may be cationic. Additional components may also be present in the skin cleansing composition (104), such as moisturizer, foaming agent, salts, preservatives, or combinations thereof.

TABLE (1)

| | % of Composition |
|---|---|
| Ingredient | (by weight) |
| Water | 87-95 |
| Cationic Surfactant | 1-5 |
| Cationic Antimicrobial Active | 0.05-0.3 |
| Cetrimonium Chloride | 0.5-2 |
| PEG-120 Methyl Glucose Dioleate | 0.1-1.5 |
| Foaming Agent | 0-1.5 |
| Humectants/Moisturizers | 0.5-3 |
| Sodium Chloride | 0.1-2.0 |
| Preservative | 0.1-1.0 |

Table (2) may present an example of a number of skin cleansing compositions (104) within the ranges disclosed in Table (1). Table (2) may present a numeric designation for each example of the skin cleansing composition (104), which may be used as a reference in comparison with the information in Tables (3), (4) and (5). Table (2) may highlight specific aspects of the formulation, which may be based on their use to determine the ratios disclosed herein, as well as additional aspects of the formulation; however, Table (2) may not disclose the entire composition of each formulation. Each formulation may also be described in terms of the weight percentage of the composition which is composed of non-aqueous components (% solids), the weight percentage of quaternary amine salt (cetrimonium chloride), the weight percentage of PEG-120 Methyl Glucose Dioleate (PEG-120), foaming agent (which may be Cocamide MEA, which may be CMEA in Table (2)), and the weight percentage of sodium chloride (NaCl). C1 and C2 in Table (2) may designate two currently marketed formulae, which may be included for comparison with the skin cleansing compositions disclosed herein. ‘Yes’ may be used in Table (2) to describe the currently marketed formulae which contain the indicated ingredient.

TABLE (2)

| | | | PEG- | | |
|---|---|---|---|---|---|
| Formulation | % Solids | Cetrimonium Cl⁻ | 120 | CMEA | NaCl |
| 1 | 8.02 | 0.75 | 0.75 | 0.00 | 1.25 |
| 2 | 7.99 | 1.34 | 1.50 | 1.50 | 0.78 |
| 3 | 8.71 | 1.87 | 0.87 | 0.90 | 1.50 |
| 4 | 9.31 | 0.50 | 1.50 | 0.00 | 0.76 |
| 5 | 8.18 | 0.75 | 1.50 | 0.00 | 0.50 |
| 6 | 9.09 | 1.34 | 0.84 | 0.84 | 1.05 |
| 7 | 8.83 | 1.70 | 1.50 | 0.00 | 0.50 |
| 8 | 8.24 | 1.38 | 0.82 | 0.00 | 0.50 |
| 9 | 6.68 | 0.50 | 0.50 | 0.00 | 0.50 |
| 10 | 8.02 | 1.50 | 0.50 | 0.00 | 0.50 |
| C1 | 7.92 | 2.30 | Yes | Yes | 0.73 |
| C2 | 7.07 | Yes | 0.00 | Yes | — |

Table (3) may compare selected rheological properties of several skin cleansing compositions (104) with those of currently marketed formulae. The comparisons in Table (3) may be on the basis of the ratios of quaternary amine salt to thickening agent (Cet.Cl⁻:PEG-120) and the ratio of quaternary amine salt to a combined mass of the non-aqueous components in the skin cleansing composition (Solids:Cet.Cl⁻). Note that the ratio of quaternary amine salt to a combined mass of the non-aqueous components may be presented in Table (3) as the ratio of the combined mass of the non-aqueous components to the quaternary amine salt. The comparisons in Table (3) may also highlight specific rheological properties, in particular the controlled shear rate test slope result (CSR Slope), which results may be presented in units of centipoise/second. A scoring for each skin cleansing composition (104) may also be shown in Table (3), wherein the rankings may constitute values for the CSR slope which are within certain ranges. A scoring for each currently marketed formulae may also be shown in Table (3), and may use the same designations as for the skin cleansing compositions (104). A lower value may indicate a more enhanced rheological property. CSR Slope values within the range of −840 to −685 centipoise/second may be given a score of 1.0; CSR Slope values within the range of −685 to −532 centipoise/second may be given a score of 2.0; CSR Slope values within the range of −532 to −379 centipoise/second may be given a score of 3.0; CSR Slope values within the range of −379 to −226 may be given a score of 4.0; CSR Slope values within the range of −226 to 0 may be given a score of 5.0.

TABLE (3)

| | | | | CSR |
|---|---|---|---|---|
| Formulation | Cet.Cl⁻:PEG-120 | Solids:Cet.Cl⁻ | CSR Slope | Score |
| 1 | 1.00 | 10.69 | −722.93 | 1.0 |
| 2 | 0.89 | 5.96 | −811.31 | 1.0 |
| 3 | 2.15 | 4.66 | −837.96 | 1.0 |
| 4 | 0.33 | 18.62 | −796.79 | 1.0 |
| 5 | 0.50 | 10.91 | −616.85 | 2.0 |
| 6 | 1.60 | 6.78 | −518.00 | 3.0 |
| 7 | 1.13 | 5.19 | −449.65 | 3.0 |
| 8 | 1.68 | 5.97 | −339.84 | 4.0 |
| 9 | 1.00 | 13.36 | −181.79 | 5.0 |
| 10 | 3.00 | 5.35 | −69.84 | 5.0 |
| C1 | — | 3.44 | −522.42 | 3.0 |
| C2 | — | — | −523.88 | 3.0 |

Table (4) may compare selected rheological properties of several skin cleansing compositions (104) with those of currently marketed formulae. The comparisons in Table (4) may be on the basis of the ratios of quaternary amine salt to thickening agent (Cet.Cl⁻:PEG-120) and the ratio of quaternary amine salt to a combined mass of the non-aqueous components in the skin cleansing composition (Solids:Cet.Cl⁻). Note that the ratio of quaternary amine salt to a combined mass of the non-aqueous components may be presented in Table (4) as the ratio of the combined mass of the non-aqueous components to the quaternary amine salt. The comparisons in Table (4) may also highlight specific rheological properties, in particular selected results of the three interval thixotropic test (3ITT). The selected results highlighted from the three interval thixotropic test may be the viscosity during the second interval (high shear stress) of the test, stated as a percentage of the original viscosity. The percent reduction in viscosity during the second interval of the three interval thixotropic test may be assigned a score, which may also be shown in Table (4). Lower scores may indicate enhanced rheological properties. The viscosity reductions in the second interval of the three interval thixotropic test within the range of 47-53% may be given a score of 1.0; those within the range of 40-46% may be given a score of 2.0; those within the range of 34-39% may be given a score of 3.0; those within the range of 26-33% may be given a score of 4.0; those within the range of 0-25% may be given a score of 5.0.

TABLE (4)

| | | | % Loss | |
|---|---|---|---|---|
| Formulation | Cet.Cl⁻:PEG-120 | Solids:Cet.Cl⁻ | in 3ITT | 3ITT Score |
| 1 | 1.00 | 10.69 | 52 | 1.0 |
| 2 | 0.89 | 5.96 | 51 | 1.0 |
| 3 | 2.15 | 4.66 | 43 | 2.0 |
| 4 | 0.33 | 18.62 | 40 | 2.0 |
| 5 | 0.50 | 10.91 | 39 | 3.0 |
| 6 | 1.60 | 6.78 | 38 | 3.0 |
| 7 | 1.13 | 5.19 | 26 | 4.0 |
| 8 | 1.68 | 5.97 | 31 | 4.0 |
| 9 | 1.00 | 13.36 | 19 | 5.0 |
| 10 | 3.00 | 5.35 | 24 | 5.0 |
| C1 | — | 3.44 | 51 | 1.0 |
| C2 | — | — | 35 | 3.0 |

Table (5) may combine the scores for each skin cleansing composition, as well as those of currently marketed formulae, to a combined score representing the overall rheological properties of the indicated skin cleansing composition. Table (5) may present both the CSR Score and the 3ITT Score (as indicated in Tables (3) and (4)). Table (5) may also present an Overall Score, which may be an average of the CSR Score and the 3ITT Score.

TABLE (5)

| Formulation | CSR Score | 3ITT Score | Overall Score |
|---|---|---|---|
| 1 | 1.0 | 1.0 | 1.0 |
| 2 | 1.0 | 1.0 | 1.0 |
| 3 | 1.0 | 2.0 | 1.5 |
| 4 | 1.0 | 2.0 | 1.5 |
| 5 | 2.0 | 3.0 | 2.5 |
| 6 | 3.0 | 3.0 | 3.0 |
| 7 | 3.0 | 4.0 | 3.5 |
| 8 | 4.0 | 4.0 | 4.0 |
| 9 | 5.0 | 5.0 | 5.0 |
| 10 | 5.0 | 5.0 | 5.0 |
| C1 | 3.0 | 1.0 | 2.0 |
| C2 | 3.0 | 3.0 | 3.0 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A skin cleansing composition with enhanced rheological properties, comprising:
a number of surfactants; and
at least one cationic antimicrobial active; and
a quaternary amine salt, wherein:
a ratio of quaternary amine salt to a combined mass of non-aqueous components in the skin cleansing composition is within the range of 1:4 to 1:15;
a ratio of quaternary amine salt to a thickening agent in the skin cleansing composition is within the range of 1:2 to 3:2;
the quaternary amine salt comprises between 0.5 and 2.0 weight percent of the skin cleansing composition; and
an aqueous medium in which the number of surfactants, the at least one cationic antimicrobial active and the quaternary amine salt are distributed.

2. The skin cleansing composition of claim 1, wherein a number of the surfactants are cationic.

3. The skin cleansing composition of claim 1, wherein a number of the surfactants are nonionic.

4. The skin cleansing composition of claim 1, further comprising a moisturizer, a foaming agent, preservative, dye, a fragrance, or combinations thereof.

5. The skin cleansing composition of claim 1, wherein the combined weight of the non-aqueous components is between 6.0% and 10.0% of the skin cleansing composition.

6. The skin cleansing composition of claim 1, wherein the quaternary amine salt comprises a cetrimonium cation and a halide anion.

7. A skin cleansing product with enhanced rheological properties, comprising:
a skin cleansing composition, wherein the skin cleansing composition includes:
a number of surfactants; and
at least one cationic antimicrobial active; and
a quaternary amine salt, wherein a ratio of quaternary amine salt to a combined mass of non-aqueous components in the skin cleansing composition is within the range of 1:4 to 1:15; and
an aqueous medium in which the number of surfactants, the at least one cationic antimicrobial active and the quaternary amine salt are distributed; and
a container to dispense the skin cleansing composition.

8. The skin cleansing product of claim 7, further comprising a thickening agent wherein a ratio of quaternary amine salt to the thickening agent in the skin cleansing composition is within the range of 1:2 to 3:2.

9. The skin cleansing product of claim 7, wherein an amount of quaternary amine salt is between 0.5 to 2.29 weight percent of the skin cleansing composition.

10. The skin cleansing product of claim 7, wherein an amount of quaternary amine salt is between 0.75 to 2.0 weight percent of the skin cleansing composition.

11. The skin cleansing product of claim 7, wherein the skin cleansing composition further comprises a moisturizer, a foaming agent, a preservative, a dye, a fragrance, or combinations thereof.

12. The skin cleansing product of claim 7, wherein the skin cleansing composition is a rinse-off formulation.

13. The skin cleansing product of claim 7, wherein the skin cleansing composition exhibits sheer thinning rheology.

14. The skin cleansing product of claim 7, wherein the skin cleansing composition exhibits a controlled shear rate test slope between −840 and −532 centipoise per second at 25 degrees Celcius (C).

15. The skin cleansing product of claim 7, wherein the skin cleansing composition loses at least 40% of its original viscosity during the second interval of a three interval thixotropic test, under a shear stress of 100 s⁻¹, at 25 degrees C.

16. A skin cleansing composition with enhanced rheological properties, comprising:
a number of surfactants;
a cationic antimicrobial active ingredient; and
an aqueous medium in which the number of surfactants and cationic antimicrobial active ingredient are distributed;
in which the skin cleansing composition possesses a controlled shear rate slope between −840 and −532 centipoise per second at 25 degrees C.; and
which loses at least 40% of its original viscosity during the second interval of a three interval thixotropic test, under a shear stress of 100 s⁻¹, at 25 degrees C.

17. The skin cleansing composition of claim 16, further comprising a quaternary amine salt.

18. The skin cleansing composition of claim 17, further comprising a thickening agent and wherein a ratio of the quaternary amine salt to the thickening agent is within the range of 1:2 to 3:2.

19. The skin cleansing composition of claim 17, wherein a ratio of the weight of the quaternary amine salt to the combined weight of all non-aqueous components is within the range of 1:4 to 1:15.

20. The skin cleansing composition of claim 17, wherein the quaternary amine salt forms between 0.5 to 2.29 weight percent of the skin cleansing composition.
